(19) United States
(12) Patent Application Publication
(10) Pub. No.: US 2009/0292310 A1
(43) Pub. Date: Nov. 26, 2009

(54) Medical device for occluding a heart defect and a method of manufacturing the same

(76) Inventors: Dara Chin, St. Paul, MN (US); Michael Corcoran, Woodbury, MN (US)

Correspondence Address: BECK AND TYSVER P.L.L.C., 2900 THOMAS AVENUE SOUTH, SUITE 100, MINNEAPOLIS, MN, 55416, US

(21) Appl. No.: 12/387,918

(22) Filed: May 8, 2009

Related U.S. Application Data
(63) Continuation-in-part of application No. 11/900,838, filed on Sep. 13, 2007.

(51) Int. Cl. A61B 17/08

(52) U.S. Cl. 606/215

(57) ABSTRACT

An implantable device for occluding a septal defect has interleaved frame sections that allow flexibility to conform to a variety of defect geometries and provide reliable occlusion during endothelialization. Left and right frames connect to opposite ends of a floating connection post. The device is resiliently deformable and is biased into a natural state wherein, in situ in a variety of defect geometries, the device applies a sandwiching force to the tissue surrounding the defect that is relatively uniform across its diameter, improving stability and promoting occlusion.

This application is a continuation-in-part of U.S. Ser. No. 11/900,838, filed Sep. 13, 2007, entitled Occlusion Device with Centering Arm Network, which is incorporated herein in its entirety.

### FIELD OF THE INVENTION

The present invention relates generally to an occlusion device for closing an aperture in a biological structure and more particularly for closing a conduit or aperture in a heart wall, such as a defect between atrial chambers.

### BACKGROUND OF THE INVENTION

The heart is comprised, generally, of four chambers: the left and right atria and the left and right ventricles. Separating the left and right sides of the heart are two walls or “septa”. The septa are susceptible to a number of types of defects, including patent ductus arteriosus, patent foramen ovale, atrial septal defects and ventricular septal defects. Although the causes and physical characteristics of these defects vary by type, they generally involve an opening (e.g. an aperture, slit, conduit, flap-covered aperture) through the septum that allows blood to shunt between chambers in the heart in an abnormal way that compromises the performance of the heart and circulatory system and has disadvantageous health consequences.

The defect in the septum can be surgically repaired via open heart surgery that requires a patient to undergo general anesthesia and requires opening of the chest cavity. Open-heart surgery is relatively risky, painful and expensive. An open-heart patient may spend several days in a hospital, will experience considerable pain, will take several weeks to recover before being able to return to normal activities, and will carry a large, prominent scar.

To avoid the risks and discomfort associated with open heart surgery, modern occlusion devices have been developed that are small, implantable devices capable of being delivered to the heart through a catheter. The delivery catheter is deployed through a relatively small incision through which it enters a major blood vessel. The catheter is snaked through the blood vessel to the heart where the occlusion device is deployed via remote (i.e. outside the body) manipulations by the doctor or cardiologist. This procedure is performed in a cardiac cathlab and avoids the risks, pain and long recovery associated with open heart surgery.

### SUMMARY OF THE INVENTION

There has been a need to improve occlusion devices to provide an easily deployable device that adapts well to a wide range of geometries, sizes, and types of defects. There has been a need for an occlusion device that centers itself within the defect, provides a reliable seal and maintains its position blocking the defect over days or weeks while the device is endothelialized (or covered by the growth of tissue). What has further been needed is an occlusion device that holds its position within the defect reliably without unduly squeezing or pinching adjacent tissue, since such squeezing can damage the tissue.

It has further been a need for the occlusion device to be retrievable so that if it is not placed initially as desired during its implantation procedure, the doctor can remove it via the catheter without damaging the device and without undue time and effort. Still further, there has been a need for an occlusion device that is easily loaded into a catheter, is easily deployed and is easily retracted back into the catheter and redeployed without removing it from the catheter for reloading so that the redeployment can be accomplished with the catheter in situ.

An occlusion device is described herein that meets these needs. The occlusion device of the present invention has left and right frames that each support a sheet. In broad terms, these left and right frames form flanges that, in situ, overlap tissue adjacent the defect and sandwich this tissue between them. A portion of the device extends through the defect.

The left frame is formed of splines that form a series of petals. These petals aid in distributing forces relatively uniformly about the periphery of the left frame.

The right frame has a set of centering limbs and a set of arms. Each limb is linked to a corresponding arm. The right sheet is coupled to the arms.

The left frame is coupled to a connecting post. The centering limbs of the right frame are also coupled to the connecting post. More specifically, the connecting post has left and right ends; the splines of the left frame are coupled to the right end of the connecting post and the limbs of the right frame are coupled to the left end of the connecting post, such that the left and right frames are interleaved or cross over one another. This arrangement yields a particularly advantageously deformable construction that allows the device to adapt to defects of a variety of sizes, shapes and configurations.

The device is resiliently deformable through a range of positions from a collapsed, delivery shape that fits within a delivery catheter to an expanded, deployed configuration, with the frame-supported sheets radiating generally outward to form flanges to sandwich tissue therebetween. The device is biased into the deployed configuration. The distance between the frame-supported sheets is variable and is determined, in situ, by the thickness of the walls of the heart adjacent the defect. The device is spring-biased toward a configuration with the frame-supported sheets immediately adjacent one another, and this bias exerts sandwiching force on the adjacent tissue. However, the device can be elongated in response to applied force to increase the distance between the sheets to accommodate varying wall thicknesses. Further, the resiliency of the frames and the manner in which they attach to the connecting post allows the frame-supported sheets to tilt with respect to one another and/or to be axially offset from one another while still reliably and effectively occluding the defect.

### BRIEF DESCRIPTION OF THE DRAWINGS

An exemplary version of an occlusion device is shown in the figures wherein like reference numerals refer to equivalent structure throughout, and wherein:

FIG. 1 is perspective view of an exemplary embodiment of an occlusion device according to the present invention;

FIG. 2 is an end view of the device of FIG. 1 taken from the right side;

FIG. 3 is an end view of the device of FIG. 1, taken from the left side, i.e. from the opposite direction of the view of FIG. 2;

FIG. 4 is a perspective view of the device of FIG. 1, under axial force;

FIG. 5 is an enlarged, partial view of the device of FIG. 1;

FIG. 6 is an enlarged schematic view of the device of FIG. 1 in situ within a heart defect;

FIGS. 7a and 7b are schematic views of the device of FIG. 1 in situ within heart defects of different wall thicknesses and showing the distribution of forces applied by the device to tissue adjacent the defect;

FIG. 8a depicts the force distribution of prior art devices on tissue adjacent a heart defect;

FIGS. 9a-9c are schematic representations of limbs of the device of FIG. 1 adapting to defects of varying cross-sectional shapes;

FIGS. 10a-c are schematic representations of the device of FIG. 1 adapting to defects of varying geometries;

FIGS. 11a-f show the device of FIG. 1 being deployed via a catheter; and

FIGS. 12a and 12b show alternate embodiments of links the connect limbs to radial arms in the device of FIG. 1

### DETAILED DESCRIPTION OF PREFERRED EMBODIMENT(S)

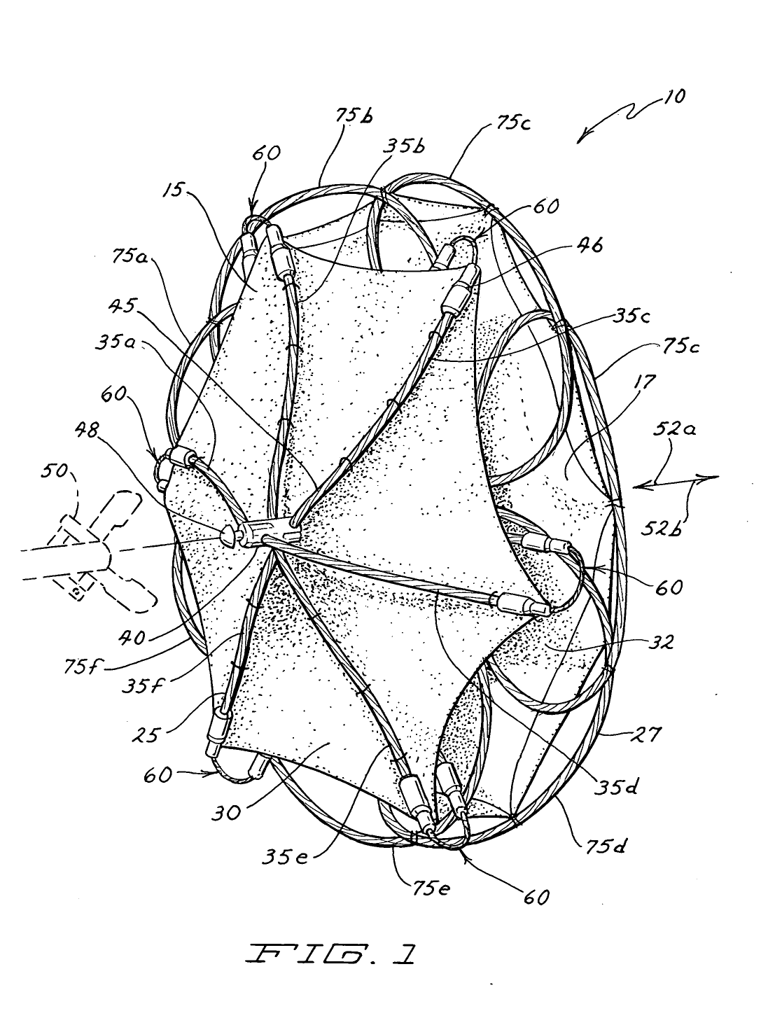

An exemplary embodiment of an occlusion device 10 is illustrated in FIG. 1. In this perspective view, the right side 15 of the device 10 is shown in the foreground and the left side 17 in the background. Throughout, the terms “right” and “left” are used for convenient reference and are selected in accord with the orientation of the device as it would typically be situated in the heart and in accord with typical cardiac terminology for distinguishing the sides of the heart. These terms should not, however, be considered limiting. (It is noted that these terms are opposite to the orientation of the device on the page in FIG. 1, such that the right side 15 of the device is on the left side of the page.) The device 10 includes right and left frames 25 and 27 respectively. A right sheet 30 is coupled to the right frame 25 and a left sheet 32 is coupled to the left frame 27.

### The Right Frame

As depicted in FIG. 1, the right frame 25 is formed in part by several radially-extending arms 35a-35f. The right frame 25 is coupled to a deployment post 40; more specifically, one end of each arm, typified by central end 45 on arm 35c, connects to the deployment post 40. The arms 35a-f radiate from the deployment post 40 and terminate at their opposite ends, typified by terminating end 46 on arm 35c, adjacent the periphery of the device 10. The deployment post 40 terminates in a grasping knob 48 that can be grasped by a deployment tool 50 that is used to exert axial forces, in the directions indicated by arrows 52a-b, to selectively deploy and retract the device 10, as will be described below.

The right sheet 30 is connected to the arms 35a-f by, for example, folding a portion (such as a tab) of the sheet around the arm. This folded-over portion can then be laminated to the frame. Alternatively, the sheet 30 can be connected to the arms 35a-f by stitches at points along the length of some or all of the arms. In this exemplary embodiment, the sheet 30 is disposed on the interior side of the arms.

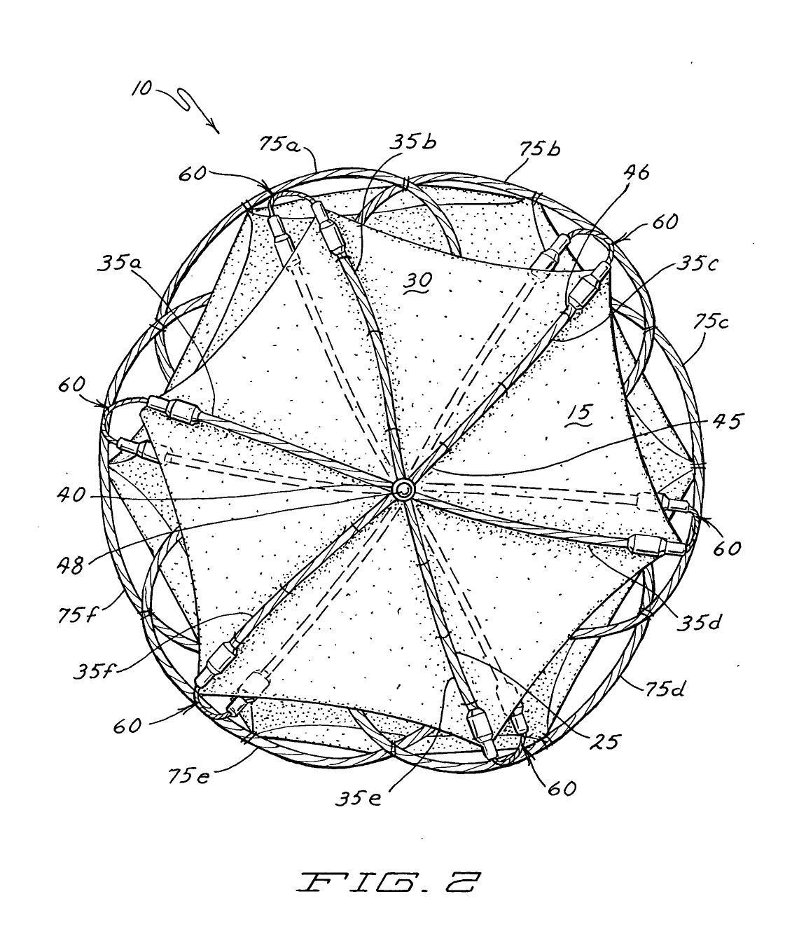

FIG. 2 shows the right frame 25 in an end view.

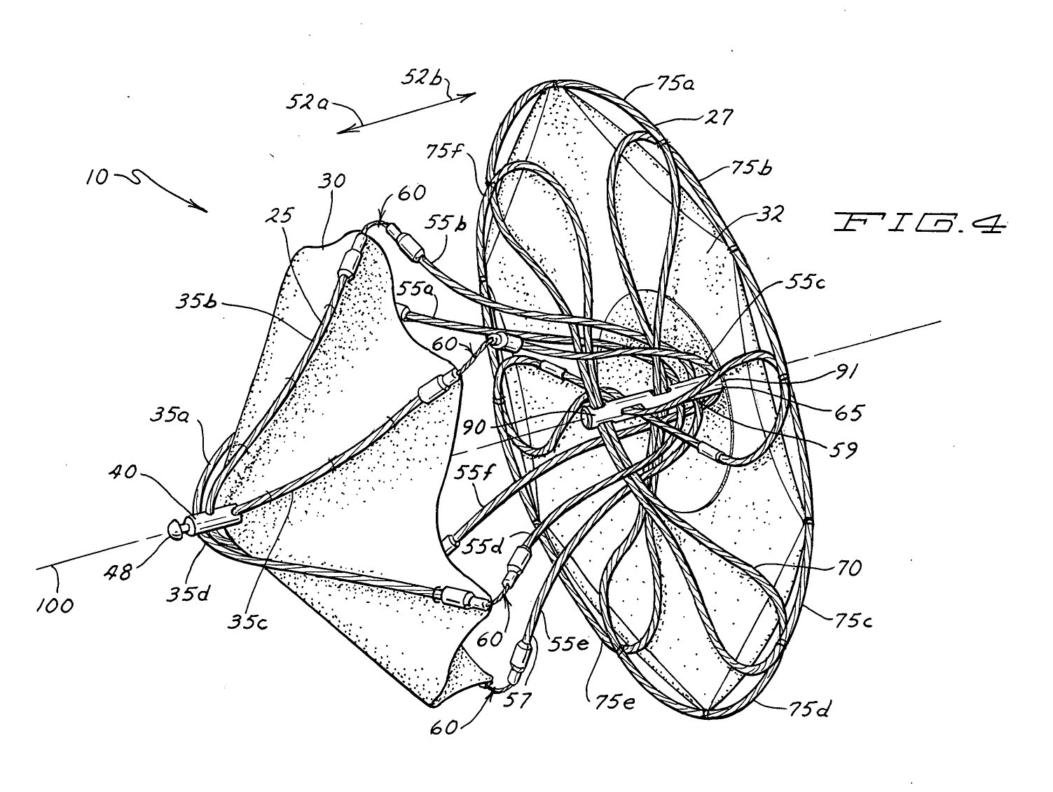

FIG. 4 reveals the structure of the device 10 between the sheets 30, 32. In addition to arms 35a-f, the right frame 25 includes elongate limbs 55a-f. These limbs 55a-f each have first and second opposite ends, typified by ends 57 and 59 on limb 55a. The limbs 55a-f are each coupled to a respective arm 35a-f via links, typified by link 60. These links 60 are couplings that allowing the limbs to fold with respect to the arms 35a-f. The links 60 will be described in greater detail below with respect to FIGS. 13a and 13b.

The opposite terminating ends 59 of the limbs 55a-f are coupled to a floating connecting post 65 in a manner that will be described in greater detail below.

### Left Frame

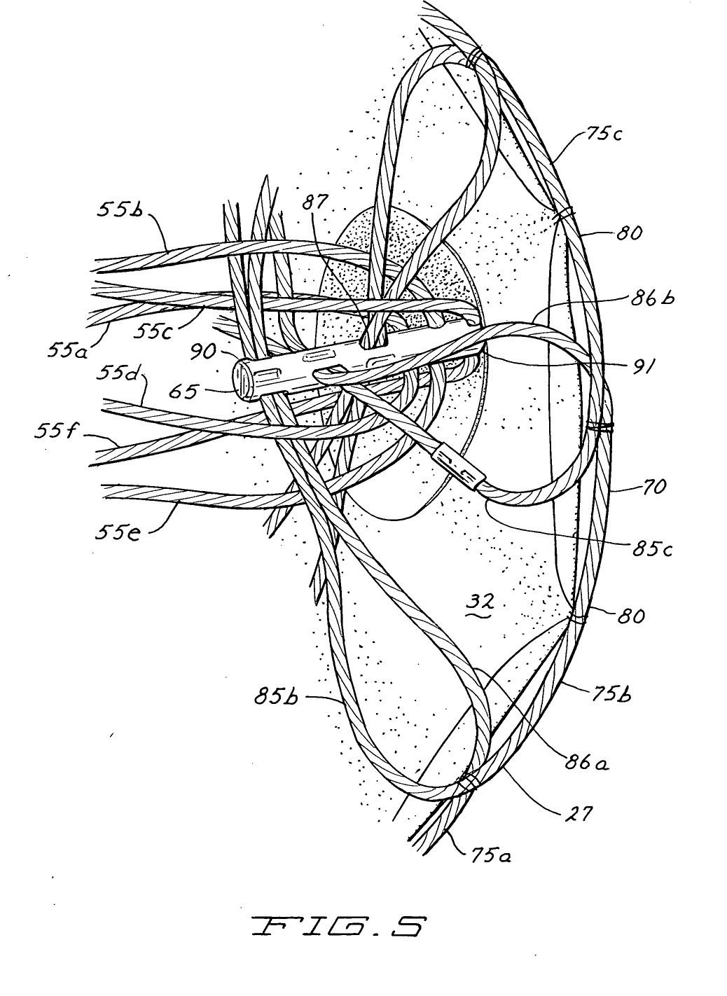

FIGS. 4 and 5 show the left frame 27 of the device 10. The left frame 27 is formed by a spline or splines 70 that form a series of overlapping loops or “petals” 75a-f that emanate or radiate from, and are coupled to, the connecting post 65. The radially outward-most portion 80 of each petal 75 defines the periphery of left frame 27. The left sheet 32 is connected to the left frame 27 by folding a portion (such as a tab) of the sheet around the frame and laminating the joint or by stitches at locations spaced about the periphery. In the exemplary embodiment illustrated, the sheet 32 is located on the exterior side of the frame 27. The petals 75a-f are interposed, such that one “edge” portion of a given petal overlaps and lies interior to the adjacent petal, while the opposite edge of the same petal overlaps and lies exterior to the opposite adjacent petal. This is apparent in FIG. 4 in which petal 75b lies between adjacent petals 75a and 75c. Left edge portion 85b of petal 75b overlaps and lies interior to right edge portion 86a of petal 75a. The right edge portion 86b of petal 75b overlaps and lies exterior to left edge portion 85c of petal 75c. This alternating over-under arrangement of adjacent petals provides stability and strength in the left frame 27, while still allowing sufficient flexibility to collapse to fit within a catheter.

The petals are formed by splines of any suitable material having the required strength and flexibility. One such suitable material is nitinol wire.

The multiple petals 75a-f of the left frame 27 can be formed of a single spline or multiple splines. In the exemplary embodiment depicted, the splines pass through apertures, typified by aperture 87, in the connecting post 65 and can be mechanically crimped to secure them. Several apertures 87 are axially spaced along the connecting post 65. Each petal is formed by a spline that exits the connecting post 65 at one location along the post's length and reenters at another location along the post's length, such that each petal is slightly askew or tilted. This aids in providing stability for the alternating over-under arrangement of adjacent petals.

The petal shapes of the splines 70 distribute forces relatively evenly about the periphery of the frame 27. This is advantageous because, in situ, the left frame 27 will not impart excessive force that would cause localized pinching or squeezing of adjacent tissue. Such pinching or squeezing at points in the tissue could prevent blood flow to the tissue and may damage the tissue. In addition, the uniform distribution of force about the periphery provides for effective and reliable occlusion, i.e. there are no locations of particularly weak force that would yield leak points. Still further, the petal shapes of the splines provide gentle curves to the periphery of the left frame 27 and that is advantageously atraumatic to tissue.

### The Connecting Post and Interleaved/Laced Frames

As shown in FIG. 4, the connecting post 65 has right and left opposite ends 90, 91, respectively. The limbs 55a-f connect to or pass through the connecting post 65 adjacent the post's left end 91; the splines 70 connect to or pass through the connecting post 65 adjacent the post's right end 90. In other words, the limbs 55a-f each connect to the connecting post 65 at positions on the post 65 that are further to the left than the positions on the post 65 to which the splines 70 connect. The result of these connecting positions is that the limbs 55a-f are laced with or are interleaved with or pass by the splines 70. One way of conceptualizing this arrangement is to imagine a plane through the post 65, perpendicular to the post's axis, between its left and right ends; both the splines 70 and the limbs 55a-f would pass through or intersect this plane. This aids in allowing the device to conform to a variety of defect geometries as will be described further below. Further, it aids in making the device easily collapsible for loading and reloading into a catheter.

Resiliency, Shape, and Range of Configurations (Natural, Deployed, in-Catheter)

Limbs 55a-f are formed of a resiliently deformable material, such as nitinol, in the form of wires or cables. In an exemplary embodiment, limbs 55a-f are subjected to pre-shaping to give them “shape memory” so that during manufacture, they are biased into a predetermined shape, even after undergoing deformation, such as when the device 10 is loaded in a catheter. One suitable shape for limbs 55a-f is a bell shape. This shape aids in allowing occlusion device 10 to maintain a low profile once the device 10 is deployed, and also allows limbs 55a-f to center the device 10 within a defect.

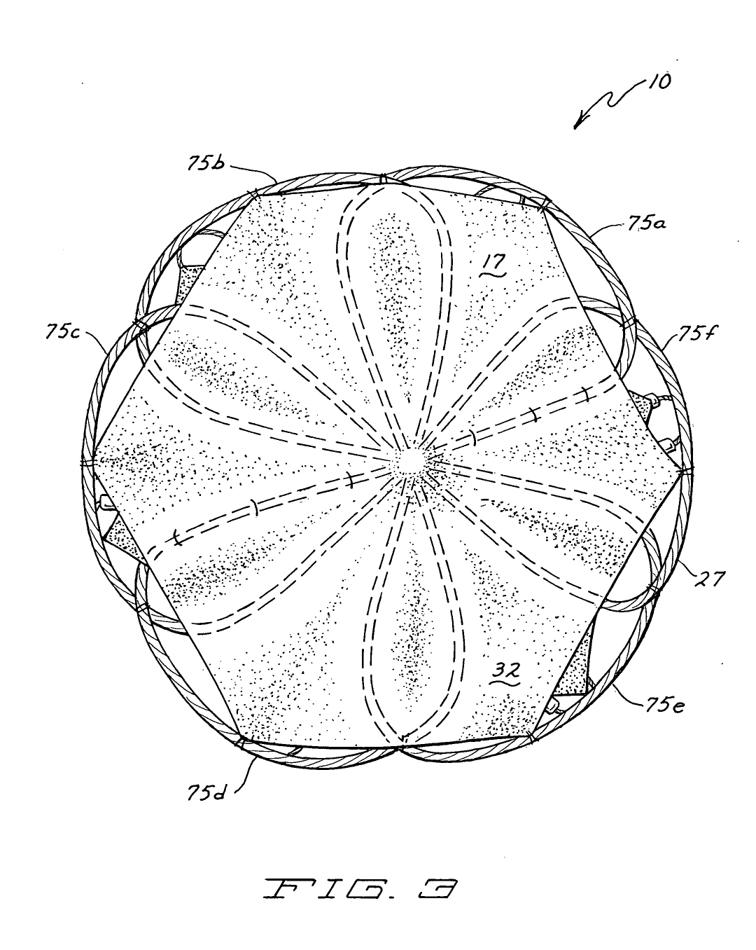

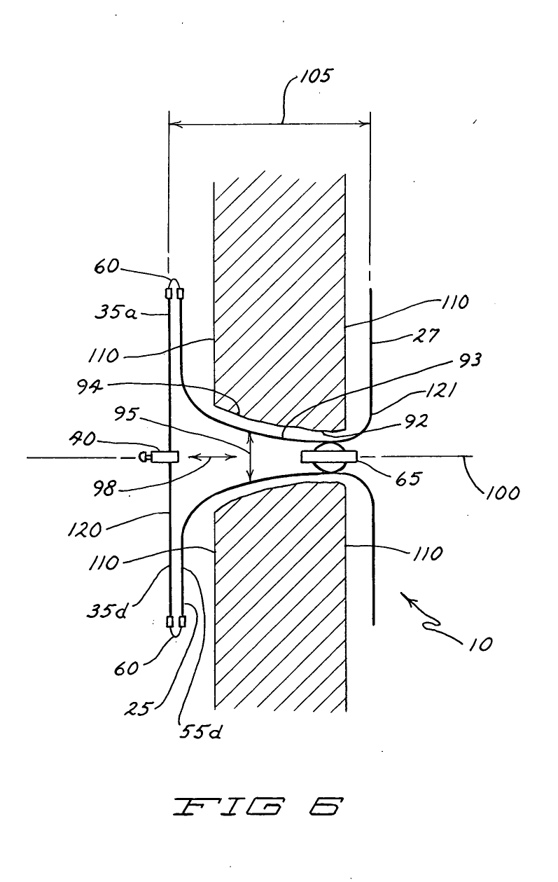

The device 10 is biased into its natural shape and configuration shown in FIGS. 1-3, in which the radial arms 35a-f of the left frame 27 extend radially outward, as do the petals 75a-f of the right frame 25, such that the arms 35a-f and the petals 75a-f form flanges 120, 121 that, in use, will sandwich tissue therebetween under slight force, as depicted in the schematic view presented in FIG. 6. Under slight axial force, the device 10 elongates slightly to accommodate tissue between the flanges 120, 121; under greater axial force, the device deforms to a collapsed configuration small enough to pass through a catheter for deployment, as will be described below. In addition, the flanges 120, 121 are constructed to provide flexibility to accommodate various defect sizes and geometries.

With further reference to FIG. 6, the device 10 positioned within a defect 92. In this in situ configuration, flanges 120, 121 are positioned on opposite sides of the defect with limbs 55a-f extending between the flanges 120, 121. More specifically, the connecting post 65 floats within the defect, and the limbs 55a-f connect thereto, as to the splines 70 of the left frame. The limbs 55a-f provide a flexible intermediate zone 93. Because the limbs 55a-f are flexible, the diameter of the intermediate zone 93 adjusts to the size and shape of the defect 92. The limbs 55a-f are biased to push outwardly to the largest diameter or periphery that the defect 92 will allow, thereby assuring that the device 10 is centered within the defect 92. (In FIG. 6, the limbs 55a and 55d are shown spaced from the tissue 94 that defines the defect 92; however, this is simply a limitation of a schematic drawing; in practice some or all of the limbs 55a-f would abut the tissue 94 adjacent the defect 92.) The biasing radially-outward force, in the direction indicated by arrow 95, supplied by the limbs 55a-f is strong enough to aid in centering the device 10 within the defect, but not strong enough to significantly displace tissue around the defect. Being properly centered increases the quality of the occlusion and thereby reduces the amount of blood that may shunt around the device 10, improving its therapeutic effect while the device 10 becomes endothelialized. Being properly centered also improves the odds of complete endothelialization.

In addition, the device 10 is resiliently deformable to allow it to increase and decrease in axial length, in the direction indicated by arrow 98 in its deployed configuration. In other words, the distance between the flanges 120, 121 or the sheets 30, 32 is varied to comply with thickness of the septum adjacent the defect. This axial length accommodation results at least in part from the flexibility in the limbs 55a-f. The limbs 55a-f move between a position in which they are roughly adjacent the center axis 100, such that the length 105 between the two sheets 30, 32 is maximized, to a position in which they splay radially outward such that the distance between the two flanges or sheets is minimized, as in FIG. 1. The limbs 55a-f are biased into the latter configuration where the distance is minimized. This bias aids the device 10 in sandwiching the tissue 110 that is adjacent the defect 89 between the flanges 120, 121 formed by the left and right frames, i.e. by exerting a force that pulls the flanges 120, 121 toward one another, thereby holding the device 10 in place until endothelialization takes place. A biased shape of the links 60, which may be resiliently deformable, may also contribute to biasing the device to its shortest axial length.

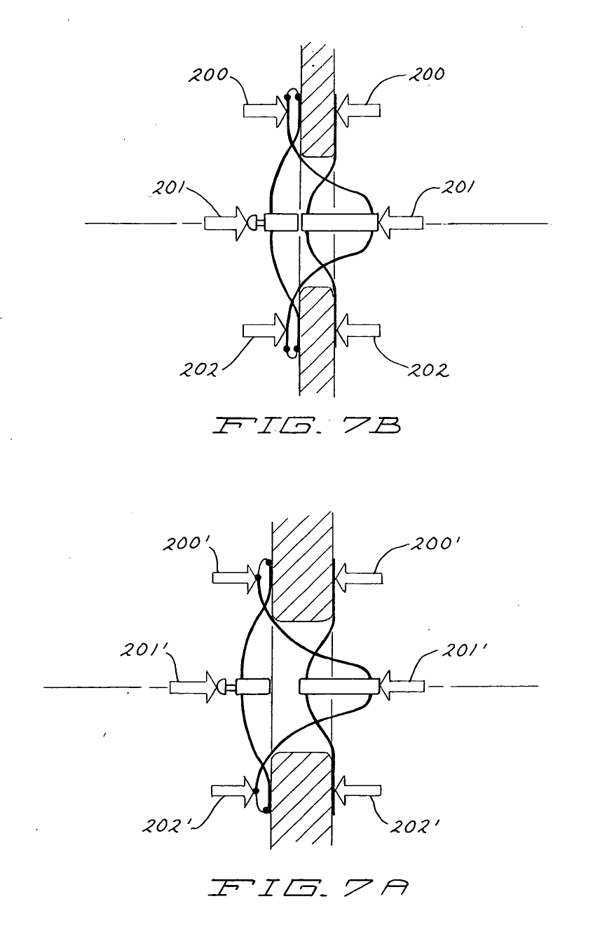

The schematics of FIGS. 7a and 7b depict the manner in which this design accommodates various wall thicknesses, as well as showing the benefits that result from the described device on force distribution on tissue adjacent the defect. The defective septum in FIG. 7a is thicker than the septum in FIG. 7b. To accommodate a thicker septum, the device 10 in FIG. 7a is expanded somewhat in its axial length. The sandwiching forces applied by the device 10 to the tissue adjacent the defect are depicted by arrows 200 and 201. More specifically, force is applied even at the radially-outermost portion of the device, aiding in holding the device 10 securely in place. Further, these forces are relatively uniform across the diameter of the device. That is, forces 200 are generally similar to forces 201 and 202. This results, in part, from the centering of the device within the defect; it results, further, from the device's natural bias, from the gentle curves of the limbs biased in a bell shape, from the interleaved configuration of the left and right frames, and from the disconnect between the post 40 to which the arms 35 are connected and the post 65 to which the splines 70 are connected allowing axial movement therebetween.

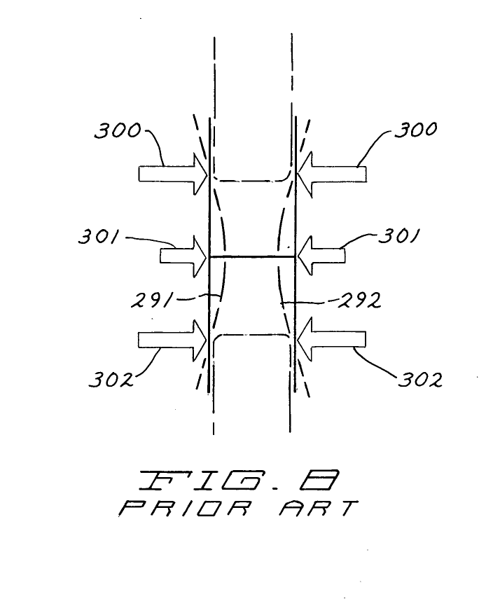

FIG. 8, in contrast, shows a prior art device that has a fixed length center post 290 extending between flanges 291, 292. The forces generated by this device are greatest at the corners of the tissue adjacent the defect. This concentration of forces 300, 302 at a particular spot in the tissue can prevent blood flow to the tissue and cause the tissue to degrade or die, thereby inhibiting occlusion. Further, the fixed post geometry offered no forces on the radial edges of flanges 291, 292, where it is most beneficial in securing the device 10.

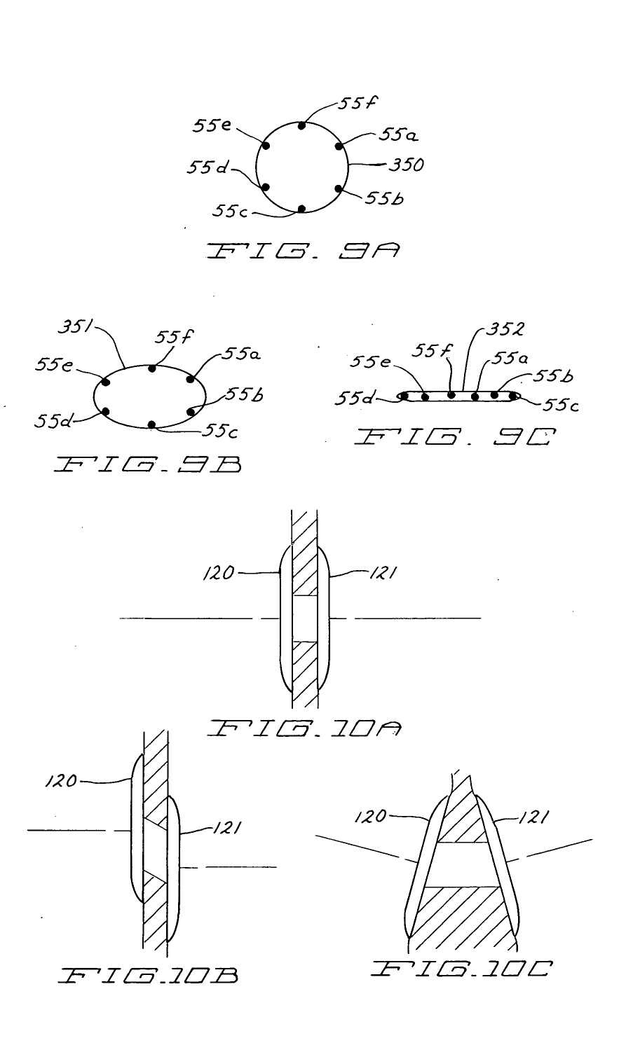

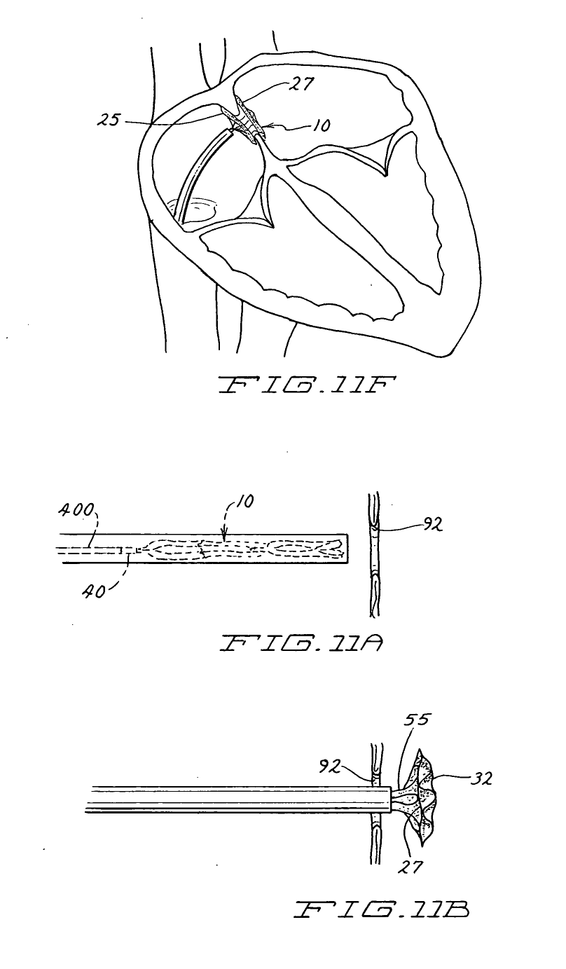

The sets of schematic drawings in FIGS. 9 and 10 show some of the flexibility and adaptability that result from the configuration of the present device 10 of FIGS. 1-4. More specifically, FIGS. 9a-9c depict a projection of the limbs 55a-f as they pass through defects of various shapes. FIG. 9a depicts a relatively circular defect 350; FIG. 9b depicts an oval-shaped defect 351; FIG. 9c depicts a defect that is very narrow or slit-shaped. The limbs 55a-f are able to conform to any of these shapes, from spreading to fill the circular shape of 350 to aligning in a single layer to fit with the slit 352.

FIGS. 10a-c further illustrate schematically how the device 10 accommodates various defect geometries. In FIG. 10b, the defect is skewed or slanted with respect to the adjacent wall; in this case, the device 10 allows for the flanges 120, 121 to similarly skew. In other words, the flanges 120, 121 have the freedom of movement to allow them to offset in their axial alignment and still provide a centered fit. FIG. 10c shows how the device 10 is able to adapt to another geometry in which the heart wall varies in thickness around the defect.

Of course, in real patients, the defects typically are defined by combinations of these alternative geometries to varying degrees and this device 10 is able to accommodate a wide range of these combinations, providing reliable occlusion where prior art devices previously did poorly or failed altogether. Further, by accommodating defects of various geometries and sizes, the device 10 yields efficiencies in manufacturing, inventory control and the like. Further, it decreases the number of devices used per procedure since the doctor need not use trial and error of a number of devices tailored to specific sizes and shapes of defects, spoiling rejected devices in the process; therefore, the cost per procedure is significantly reduced. Nevertheless, it is possible to tailor the device more particularly to various defect shapes and sizes by heat-shaping the limbs 55a-f accordingly.

### Deployment

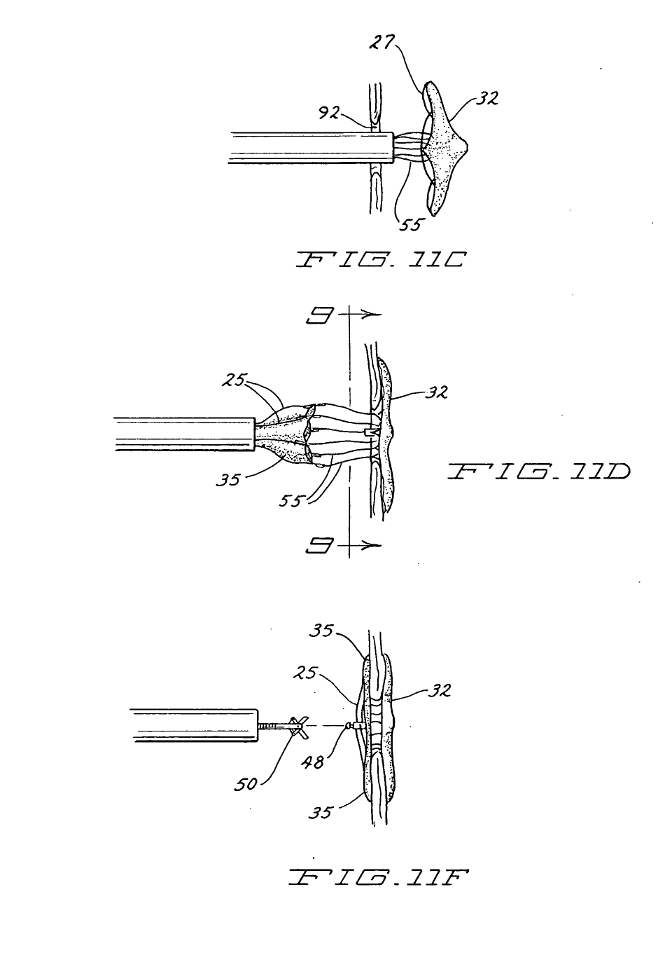

As noted, the device 10 can, under axial force, deform to a collapsed configuration to fit within a catheter for delivering the device to the defect site. FIGS. 11a-f depict in series how the device 10 is deployed. As shown in FIG. 11a, the device 10 in its collapsed state within a catheter and connected to a deployment wire 400 connected to a deployment post 40, is maneuvered into position adjacent the defect to be occluded. As depicted in FIG. 11b, the terminating end of the catheter is positioned on the opposite side of the defect 92. The device 10 is pushed partway out of the catheter, so that the left frame exits the catheter. The left frame, freed from the catheter, expands to its naturally-biased shape as shown in FIG. 11c. The operator snugs the left frame against the heart wall adjacent the defect and then continues to expel the device from the catheter, FIG. 11d. When the right frame is freed from the catheter, it adopts its naturally-biased configuration, shown in FIG. 11e. The operator disconnects the deployment wire 400 from the device 10, as shown in FIG. 11f.

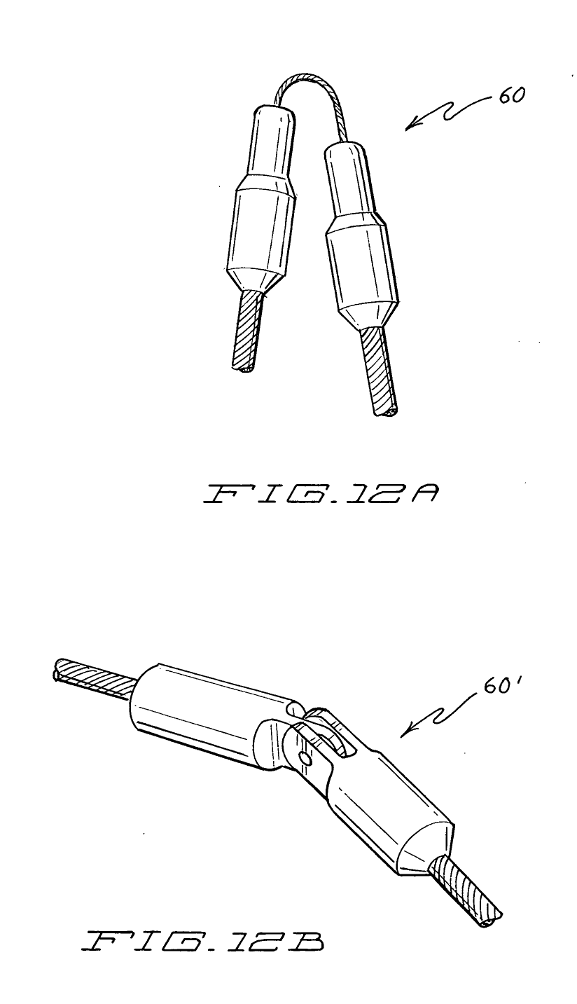

Although an illustrative version of the device is shown, it should be clear that many modifications to the device may be made without departing from the scope of the invention. For example, two exemplary embodiments of the links 60, 60′ are depicted in FIGS. 12a and 12b. In an exemplary embodiment of FIG. 12a, a link 60 are made of a relatively small-diameter wire to provide for a relatively sharp, or small radius-of-curvature, bend. In the exemplary embodiment of FIG. 12b, the link 60′ is a hinge about an axis. In an alternative embodiment of a link not shown, associated limbs and arms might each be formed of a unitary member with a transition region between the limb portion and the arm portion that may have different strength or flexibility properties than the limb and arm portions. By joining the arms and limbs via links or transition regions, optimal choices can be made to provide the desired strength in the limbs and arms while achieving flexibility in the joints or transition therebetween.

I claim:

1. A device for occluding a defect in a heart wall, comprising:
a) a left frame;
b) a right frame;
c) a left sheet coupled to said left frame;
d) a right sheet coupled to said right frame;
e) a connecting post having left and right ends;
f) said right frame coupled to said connecting post adjacent the post's left end and said left frame coupled to said connecting post adjacent the post's right end, such that said left and right frames are interleaved.

2. A device according to 1 wherein said right frame is resiliently deformable and is biased toward a first deployed configuration in which said left and right sheets are in close proximity and further wherein said right frame is deformable under applied force to elongate thereby distancing said right frame from said left frame under tension to accommodate heart walls of various thickness and to squeeze heart wall tissue adjacent the defect slightly to hold said device in place.

3. A device according to claim 2 wherein said right frame has elongate interior limbs coupled to exterior radial arms and wherein said right sheet is coupled to said radial arms.

4. A device according to claim 1 wherein said limbs are resiliently deformable to comply with the shape of a defect in which the device is positioned.

5. A device according to claim 4 wherein said limbs are resiliently deformable to comply with a slot-shaped defect with said limbs,

6. A device according to claim 1 wherein said left and right frames are each resiliently deformable between a deployed, expanded configuration and a collapsed, delivery configuration and wherein said device is biased toward said expanded configuration.

7. A device according to claim 6 wherein said frames expand independently of one another such that either one of said frames can be in a deployed configuration while the other said frame is in a delivery configuration.

8. A device according to claim 6, wherein, in said biased, expanded configuration said left and right sheets are slightly concave in the same direction, such that they tend to nest.

9. A device for occluding a defect in a heart wall, comprising:
a) a left frame;
b) a right frame;
c) a connecting post coupled to said left and right frames;
d) wherein said left frame is formed of splines arrayed in a series of petals.

10. A device for occluding a defect in a heart wall according to claim 9 wherein adjacent petals overlap.

11. A device according to 9 wherein said splines are arrayed in a series of six petals.
